(19) United States
(12) Patent Application Publication
(10) Pub. No.: US 2025/0352438 A1
(43) Pub. Date: Nov. 20, 2025

(54) INVERSE EMULSION FOR CARING FOR THE SKIN

(71) Applicant: L'OREAL, Paris (FR)

(72) Inventors: Anne BOUCHARA, CHEVILLY LARUE (FR); Jean-Christope SALVI, CHEVILLY LARUE (FR)

(21) Appl. No.: 18/857,397

(22) PCT Filed: Apr. 18, 2023
(86) PCT No.: PCT/EP2023/060037

§ 371 Date: Oct. 16, 2024

(30) Foreign Application Priority Data
Apr. 19, 2022 (FR) FR2203610

(51) Int. Cl. A61K 8/02; A61K 8/44; A61K 8/73; A61K 8/96; A61Q 19/00

(52) U.S. Cl. CPC A61K 8/025; A61K 8/442; A61K 8/731; A61K 8/965; A61Q 19/00

(57) ABSTRACT

The present invention relates to a composition, preferably a cosmetic composition, of the inverse emulsion type, particularly free of microplastic particles, and comprising: (i) one or more spherical filler(s) having a mean diameter ranging from 1 to 100 μm; (ii) one or more filler(s) having an oil absorption capacity, measured at the wet point, of greater than 10 ml/g of fillers; and (iii) one or more native or modified starch(es). The invention also relates to a cosmetic process for caring for and/or cleansing keratin materials, in particular the skin, comprising the application, to said keratin materials, of a composition according to the invention.

### TECHNICAL FIELD

The present invention aims to provide a novel composition, preferably a cosmetic composition, for the care and/or hygiene of keratin materials, particularly of the skin of the face and/or of the body.

It aims more particularly to provide an alternative to the presence of microplastic particles in compositions of the inverse emulsion type, particularly for caring for keratin materials, while retaining the textural and sensorial properties that these particles afford such compositions.

### PRIOR ART

One of the major challenges for developers of cosmetic compositions is meeting the demands of consumers in terms of lightness of texture and sensoriality (soft feel), in particular for compositions for caring for the skin, and particularly facial skin.

Thus, it is common to introduce microplastic particles, which are particularly very good textural and sensorial agents, into cosmetic compositions for caring for keratin materials, such as creams, shower gels or foundations. Moreover, another consumer demand nowadays is that of having environmentally-friendly cosmetic products, in particular natural and/or eco-friendly products, i.e. products whose design and development take into account their environmental impacts.

As such, consumers and developers of cosmetic compositions wish to be freed as far as possible from “chemical” products, favouring “natural” products which advantageously do not persist in the environment. For obvious reasons, it is therefore now being sought to do away with materials such as the abovementioned microplastics which contribute, even on a small scale, to the pollution of water with plastics, and thus contaminate not only the water itself but also the entire marine ecosystem. The UNEP (United Nations Environment Programme) estimated, as early as 2005, that each km² of ocean contained 13 000 plastic particles.

### DISCLOSURE OF THE INVENTION

Thus, there is a need to have cosmetic compositions, particularly of the inverse emulsion type, that do not comprise microplastic particles while retaining satisfactory textural and sensorial properties for consumers, in particularly properties that are at least equivalent to those of the corresponding cosmetic compositions that do comprise microplastic particles. In particular, there is a need to have compositions, particularly of the inverse emulsion type, for caring for and/or cleansing keratin materials, in particular the skin, particularly facial or body skin, which are free of microplastic particles and have sensorial properties, particularly in terms of appearance and handling of the cream (shine, smoothness, transparency, fluidity, wobble, tack on the fingers, handling, consistency, shape holding when handled, and thickness), in terms of application qualities (whiteness of the film, greasy feel) and in terms of skin finish, that are identical to those of a corresponding composition comprising microplastic particles.

However, at the current time there are no non-microplastic fillers that have the same characteristics as microplastic particles.

Finally, the need also remains to have compositions that are compatible with the current consumer demands, particularly regarding the environment.

The present invention is specifically directed towards meeting these needs.

### SUMMARY OF THE INVENTION

Thus, according a first aspect thereof, the present invention relates to a composition, preferably cosmetic composition, particularly for caring for or cleansing keratin materials, in particular the skin, more particularly facial skin, comprising:
i) one or more spherical filler(s) which are organic or inorganic, in particular organic, having a mean diameter ranging from 1 to 100 μm;
ii) one or more filler(s) which are in particular organic, having an oil absorption capacity, measured at the wet point, of greater than 10 ml/g of fillers, said fillers being separate from the fillers i); and
iii) one or more native or modified starch(es);

said composition being an inverse emulsion.

The inventors have observed, surprisingly, that the combination of spherical filler(s) having a mean diameter ranging from 1 to 100 μm, of separate filler(s) having an oil absorption capacity, measured at the wet point, of greater than 10 ml/g of fillers, and of native or modified starch(es), makes it possible to obtain, in compositions of inverse emulsion type, sensorial properties, particularly in terms of appearance and handling of the cream (shine, smoothness, transparency, fluidity, wobble, tack on the fingers, handling, consistency, shape holding when handled, and thickness), in terms of application qualities (whiteness of the film, greasy feel) and in terms of skin finish, that are identical to a corresponding composition comprising microplastic particles.

A composition according to the invention can thus comprise less than 0.1% by weight relative to the total weight of the composition, in particular less than 0.05% by weight, particularly less than 0.01% by weight, and more particularly 0% by weight of microplastic particles.

A composition according to the invention can comprise at least 0.05% by weight, in particular from 0.05% to 2% by weight, more particularly from 0.05% to 1% by weight, particularly from 0.05% to 0.5% by weight, in particular from 0.05% to 0.2% by weight, and more particularly from 0.1% to 0.2% by weight of spherical fillers i) relative to the total weight of the composition.

A composition according to the invention can further comprise at least 0.1% by weight, in particular from 0.1% to 2% by weight, particularly from 0.1 to 1% by weight, and more particularly from 0.2% to 0.8% by weight of fillers ii) relative to the total weight of the composition.

A composition according to the invention can moreover comprise at least 1% by weight, in particular from 1% to 5% by weight, particularly from 1.5% to 4% by weight, and more particularly from 2% to 4% by total weight of (native and/or modified) starch relative to the total weight of the composition.

In particular, in a composition according to the invention, the spherical fillers i) can be selected from the group consisting of cellulose beads; glass microbeads; silica microbeads, in particular porous silica microbeads or amorphous silica microbeads; N-lauroyl L-lysine, and perlite, in particular expanded perlite; in particular selected from the group consisting of cellulose beads; glass microbeads; N-lauroyl L-lysine, and perlite, in particular expanded perlite; and are more particularly selected from the group consisting of cellulose beads and perlite.

In particular, in a composition according to the invention, the fillers ii) can be silica aerogel particles and in particular hydrophobic silica aerogel particles, particularly are particles of silica aerogel treated with hexamethyldisiloxane.

In particular, in a composition according to the invention, the native or modified starch may be different from the filler(s) (ii), in particular from the filler(s) (i) and the filler(s) (ii). In particular, in a composition according to the invention, the native or modified starch can be selected from starches of corn, rice, cassava, potato, wheat, sorghum, pea, and mixtures thereof, and is in particular corn starch.

Moreover, in a composition according to the invention, the modified starch can be selected from pre-cooked starches; hydrolysed starches; crosslinked starches, for example crosslinked by a methylurea derivative, by octenylsuccinic anhydride or by epichlorohydrin; esterified starches; etherified starches; oxidised starches; refined starches; starches grilled in the presence of acid; grafted starches, for example grafted with sodium polyacrylates; coated starches, for example coated with amino acids; and mixtures thereof, preferably selected from crosslinked starches, and particularly starches crosslinked by octenylsuccinic anhydride.

In particular, a composition according to the invention can be such that the ratio between the content of filler(s) ii) and the content of spherical fillers i) is between 0.1 and 15, in particular between 0.5 and 8, and preferably between 1 and 3.

In particular, a composition according to the invention can be such that the ratio between the content of filler(s) ii) and the sum of the contents of spherical fillers i) and of native or modified starch(es) is less than 1.

A composition according to the invention can in particular comprise:
i) at least 0.05% by weight, relative to the total weight of the composition, in particular from 0.05% to 2% by weight, more particularly from 0.05% to 1% by weight, particularly from 0.05% to 0.5% by weight, in particular from 0.05% to 0.2% by weight, and more particularly from 0.1% to 0.2% by weight of spherical filler(s) having a mean diameter ranging from 1 to 100 μm, in particular ranging from 1 to 50 μm, more particularly less than 30 μm, and particularly of between 1 and 25 μm, selected from the group consisting of cellulose beads and perlite;
ii) at least 0.1% by weight, relative to the total weight of the composition, in particular from 0.1% to 2% by weight, particularly from 0.1% to 1% by weight, and more particularly from 0.2% to 0.8% by weight of filler(s) having an oil absorption capacity, measured at the wet point, of greater than 10 ml/g of fillers, said fillers being particles of silica aerogel treated with hexamethyldisiloxane;
iii) at least 1% by weight, in particular from 1% to 5% by weight, particularly from 1.5% to 4% by weight, and more particularly from 2% to 4% by total weight of native or modified starch(es), the starch being native or modified corn starch, in particular modified corn starch, more particularly corn starch crosslinked by octenylsuccinic anhydride; and
iv) less than 0.1% by weight, in particular less than 0.05% by weight, particularly less than 0.01% by weight, and more particularly 0% by weight of microplastic particles relative to the total weight of the composition.

Moreover, the present invention also relates to a cosmetic process for caring for and/or cleansing keratin materials, in particular the skin, and more particularly facial skin, comprising the application, to said keratin materials, of a cosmetic composition in accordance with the invention.

In the context of the present invention, and unless otherwise indicated, the following definitions apply:

For the purposes of the present invention, the term “fillers” should be understood as meaning colourless or white, mineral or organic, natural or synthetic solid particles of any form, which are in an insoluble and dispersed form in the medium of the composition.

According to the invention, the term “keratin materials” is intended to denote the skin of the body, of the face and/or of area around the eyes, the lips, the nails, the mucous membranes, the eyelashes, the eyebrows, body hair, the beard, the scalp and/or head hair, or any other cutaneous area of the body. More particularly, the keratin materials according to the invention are the scalp, head hair and/or the skin.

The term “skin” is intended to mean all of the skin of the body, and preferably the skin of the face, neckline, neck, arms and forearms, or more preferably still the skin of the face, in particular of the forehead, nose, cheeks, chin and area around the eyes.

The compositions according to the present invention are preferably cosmetic compositions. For the purposes of the present invention, the term “cosmetic” is intended to denote a composition compatible with the skin, mucous membranes and skin appendages.

A composition according to the invention is suitable for application to keratin materials, in particular topical application to the skin, and thus comprises a physiologically acceptable medium, i.e. a medium that is compatible with the skin.

It is preferably a cosmetically acceptable medium, i.e. a medium which has a pleasant colour, odour and feel and which does not cause any unacceptable discomfort, i.e. stinging, tautness or redness, liable to discourage the user from applying this composition.

The term “at least one” is equivalent to “one or more”.

The terms “between . . . and . . . “, “comprises from . . . to . . . “, “formed from . . . to . . . ” and “ranging from . . . to . . . ” should be understood as being inclusive of the limits, unless otherwise specified.

### DETAILED DESCRIPTION

### Water-In-Oil Emulsion or Inverse Emulsion

A composition according to the invention is a water-in-oil emulsion, referred to as an inverse emulsion. An inverse emulsion is generally constituted of an oily liquid phase, in this case the continuous phase, and of an aqueous liquid phase, in this case the dispersed phase. It is a dispersion of droplets of the aqueous phase in the oily phase.

### Spherical Filler of a Composition According to the Invention

A composition in accordance with the invention comprises one or more spherical filler(s), these fillers having a mean diameter ranging from 1 to 100 μm.

The term “spherical” is intended to mean that the particle has a sphericity index, i.e. the ratio between its largest diameter and its smallest diameter, of less than 1.2.

The term “mean particle size” means the mean diameter on 50% by volume of the particles (D[0,5]) obtained using a laser diffraction particle size analyser (e.g. Mastersizer 2000 from the company Malvern). It is understood that the term “particle” can for example designate, as apparent below, the fillers of a composition according to the invention, and in particular the spherical filler(s) as detailed below.

In the present text, these fillers are also sometimes referred to by the name “spherical fillers i)”.

In particular, the mean diameter of spherical filler(s) in accordance with the invention may be between 1 and 50 μm, is less than 30 μm, and is in particular between 1 and 25 μm. A composition according to the invention may comprise at least 0.05% by weight of spherical fillers in accordance with the invention, relative to the total weight of the composition, and can particularly comprise from 0.05% to 2% by weight, more particularly from 0.05% to 1% by weight, particularly from 0.05% to 0.5% by weight, in particular from 0.05% to 0.2% by weight, and more particularly from 0.1% to 0.2% by weight of spherical fillers i) relative to the total weight of the composition.

The spherical fillers may be inorganic or organic, preferably organic.

A spherical filler in accordance with the invention may be selected from the group consisting of cellulose beads; glass microbeads; silica microbeads, in particular porous silica microbeads or amorphous silica microbeads; N-lauroyl L-lysine, and perlite, in particular expanded perlite.

A spherical filler in accordance with the invention may more particularly be selected from the group consisting of cellulose beads; glass microbeads; N-lauroyl L-lysine, and perlite, in particular expanded perlite.

A spherical filler in accordance with the invention may more particularly be selected from the group consisting of cellulose beads and perlite, in particular expanded perlite.

The spherical fillers can thus be selected from spherical cellulose beads and/or microcrystalline celluloses in the form of spheres, and more preferentially from spherical cellulose beads. The cellulose beads which can be used are not limited by the type of cellulose, such as cellulose I, cellulose II or the like.

The spherical fillers of the present invention may be porous or nonporous.

As spherical fillers which are suitable for the invention, mention may for example be made, by way of illustration, of those indicated in Table 1 below.

TABLE 1

| Customary | | | | | |
|---|---|---|---|---|---|
| chemical name | Particle size | | Chemical family | Trade name | Supplier |
| SPHERICAL | 4-7 | μm | Carbohydrates | Cellulobeads USF | KOBO |
| CELLULOSE | | | | | |
| BEADS | | | | | |
| SOLID | <15 | μm | Carbohydrates | Cellulobeads D- | DAITO KASEI |
| CELLULOSE | | | | 10 | KOGYO |
| BEADS | | | | | |
| SOLID | <30 | μm | Carbohydrates | Cellulobeads D- | DAITO KASEI |
| CELLULOSE | | | | 30 | KOGYO |
| BEADS | | | | | |
| SOLID | <100 | μm | Carbohydrates | Cellulobeads D- | OMYA |
| CELLULOSE | | | | 100 | |
| BEADS | | | | | |
| GLASS | 9-11 | μm | Silica | P2015SL | PRIZMALITE |
| MICROBEADS | | | | | |
| SILICA | 3-15 | μm | Silica | Spherica P-1500 | JGC CATALYSTS |
| MICROBEADS | | | | | & CHEMICALS |
| POROUS SILICA | 1-16 | μm | Silica | Silica bead SB- | MIYOSHI KASEI |
| MICROBEADS | | | | 300 | |
| AMORPHOUS | 5 | μm | Silica | Sunsphere H51 | AGC SI-TECH |
| SILICA | | | | | |
| MICROSPHERES | | | | | |
| AMORPHOUS | 8 | μm | Silica | Chiffonsil P-3R | JGC CATALYSTS |
| SILICA | | | | | & CHEMICALS |
| MICROBEADS | | | | | |
| N-LAUROYL L- | 10-20 | μm | Modified amino | Amihope LL | AJINOMOTO |
| LYSINE | | | acid | | |
| EXPANDED | 25 | μm | Perlite | Optimat 2550 | IMERYS |
| PERLITE | | | | OR | |

Filler Having an Oil Absorption Capacity, Measured at the Wet Point, of Greater than 10 ml/g of Fillers of a Composition According to the Invention

A composition in accordance with the invention comprises one or more filler(s) having an oil absorption capacity, measured at the wet point, of greater than 10 ml/g of fillers. In the present text, these fillers are also sometimes referred to by the name “fillers ii)”. These fillers are separate from the spherical fillers, or spherical fillers i), in accordance with the invention.

These fillers are moreover preferably separate from waxy particles.

The oil absorption capacity, measured at the wet point and denoted Wp, corresponds to the amount of oil that needs to be added to 100 g of particles in order to obtain a homogeneous paste. It is measured according to the “wet point” method or the method for determining the oil uptake of a powder according to the principle described in standard NF T 30-022. It corresponds to the amount of oil adsorbed onto the available surface of the powder and/or absorbed by the powder by measuring the wet point, described below:

An amount m=2 g of powder is placed on a glass plate and then the oil (oleic acid) is added dropwise. After addition of 4 to 5 drops of oil to the powder, mixing is performed using a spatula, and addition of oil is continued until conglomerates of oil and powder have formed. From this point, the oil is added at the rate of one drop at a time and the mixture is subsequently triturated with the spatula. The addition of oil is stopped when a firm, smooth paste is obtained. This paste must be able to be spread over the glass plate without cracks or the formation of lumps. The volume Vs (expressed in ml) of oil used is then noted. The oil uptake (oil absorption capacity) corresponds to the ratio Vs/m.

The fillers (ii) of a composition according to the invention are in particular inorganic fillers. A composition according to the invention can comprise at least 0.1% by weight, in particular from 0.1% to 2% by weight, more particularly from 0.1% to 1.5% by weight, particularly from 0.1% to 1% by weight, and more particularly from 0.2% to 0.8% by weight of fillers ii) relative to the total weight of the composition.

The fillers ii) in accordance with the invention may have a size, expressed as the mean diameter (D [0,5]), of less than 50 μm, in particular ranging from 1 to 30 μm, more particularly from 5 to 25 μm, and particularly from 5 to 15 μm.

The sizes of the aerogel particles according to the invention may be measured by static light scattering using a commercial particle size analyser of the MasterSizer 2000 type from Malvern. The data are processed on the basis of the Mie scattering theory. This theory, which is exact for isotropic particles, makes it possible to determine, in the case of non-spherical particles, an “effective” particle diameter. This theory is notably described in the publication by Van de Hulst, H. C., Light Scattering by Small Particles, Chapters 9 and 10, Wiley, New York, 1957.

The fillers ii) in accordance with the invention may be silica aerogel particles and in particular hydrophobic silica aerogel particles.

The term “hydrophobic silica” means any silica, the surface of which is treated with silylating agents, for example with halogenated silanes such as alkylchlorosilanes, siloxanes, in particular dimethylsiloxanes such as hexamethyldisiloxane, or silazanes, so as to functionalize the OH groups with silyl groups Si-Rn, for example trimethylsilyl groups. The preparation of hydrophobic silica aerogel particles surface-modified by silylation is well known and is for example described more fully in U.S. Pat. No. 7,470,725.

The fillers ii) in accordance with the invention may particularly be particles of silica aerogel treated with hexamethyldisiloxane.

Mention can thus be made, as a filler ii) in accordance with the invention, of the aerogel sold under the name DOWSIL VM-2270 AEROGEL FINE PARTICLES (INCI name: Silica silylate), by Dow Corning.

### Starch of a Composition According to the Invention

A composition according to the invention further comprises one or more starch(es), these starch(es) being native or modified.

The starches that may be used in the present invention are more particularly macromolecules in the form of polymers consisting of elementary moieties which are anhydroglucose units (dextrose), linked via α(1,4) bonds, of chemical formula C₆H₁₀O₅. The number of these moieties and their assembly make it possible to distinguish amylose, a molecule formed from approximately 600 to 1000 linearly linked glucose molecules, from amylopectin, a polymer branched approximately every 25 glucose residues (a (1-6) bond). The total chain may include between 10 000 and 100 000 glucose residues.

Starch is described in particular in “Kirk-Othmer's Encyclopedia of Chemical Technology, 3rd edition, volume 21, pages 492-507, Wiley Interscience, 1983”. The relative proportions of amylose and of amylopectin, and their degree of polymerization, vary as a function of the botanical origin of the starches. On average, a sample of native starch is constituted of approximately 25% amylose and 75% amylopectin. Occasionally, phytoglycogen is present (between 0% and 20% of the starch), which is an analogue of amylopectin but branched every 10 to 15 glucose residues. Starch may be in the form of semicrystalline granules: amylopectin is organized in sheets, amylose forms a less well organized amorphous zone between the various sheets. Amylose is organized in a straight helix with six glucoses per turn. It dissociates into assimilable glucose under the action of enzymes, amylases, all the more easily when it is in amylopectin form. Specifically, the helical formation does not promote the accessibility of the starch to the enzymes.

Starches are generally in the form of a white powder, which is insoluble in cold water, of which the elemental particle size ranges from 3 to 100 microns. By treating it with hot water, starch paste is obtained. It is exploited in industry for its thickening and gelling properties.

The botanical origin of the starch molecules used in the present invention may be cereals or tubers. In particular, the starch in accordance with the invention may be derived from any plant source of starch, particularly such as corn, rice, cassava, potato, wheat, sorghum or pea. It is also possible to use hydrolyzates of the starches mentioned above.

The native starches are represented, for example, by the products sold under the names C*Amilogel™, Cargill Gel™, C* Gel™, Cargill Gum™, DryGel™, C*Pharm Gel™ by Cargill, under the name Amidon de Maïs by Roquette, and under the name Tapioca Pure by National Starch.

The modified starches which can be used in a composition of the invention may be starch molecules modified via one or more of the following reactions: pregelatinization, degradation (acid hydrolysis, oxidation, dextrinization), substitution (esterification, etherification), crosslinking (esterification), bleaching.

More particularly, these reactions may be performed in the following manner:
pregelatinization by splitting the starch granules (for example drying and cooking in a drying drum);
acid hydrolysis giving rise to very rapid retrogradation on cooling;
oxidation with strong oxidizing agents (alkaline medium, in the presence of sodium hypochlorite NaOCl for example) leading to the depolymerization of the starch molecule and to the introduction of carboxyl groups into the starch molecule (mainly oxidation of the hydroxyl group at C₆);
dextrinization in acid medium at high temperature (hydrolysis followed by repolymerization);
crosslinking with functional agents capable of reacting with the hydroxyl groups of the starch molecules, which will thus be bonded together (for example with glyceryl and/or phosphate groups);
esterification in alkaline medium for the grafting of functional groups, particularly C₁-C₆ acyl (acetyl), C₁-C₆ hydroxyalkyl (hydroxyethyl or hydroxypropyl), carboxymethyl or octenylsuccinic.

Monostarch phosphates (of the type Am-O-PO-(OX)2), distarch phosphates (of the type Am-O-PO-(OX)-O-Am) or even tristarch phosphates (of the type Am-O-PO-(O-Am)2) or mixtures thereof may particularly be obtained by crosslinking with phosphorus compounds. X particularly denotes alkali metals (for example sodium or potassium), alkaline-earth metals (for example calcium or magnesium), ammonium salts, amine salts, for instance those of monoethanolamine, diethanolamine, triethanolamine, 3-amino-1,2-propanediol, or ammonium salts derived from basic amino acids such as lysine, arginine, sarcosine, ornithine or citrulline.

The phosphorus compounds may be, for example, sodium tripolyphosphate, sodium orthophosphate, phosphorus oxychloride or sodium trimetaphosphate.

A native or modified starch in accordance with the invention can be selected from starches of corn, rice, cassava, potato, wheat, sorghum, pea, and mixtures thereof, and is in particular corn starch.

A starch in accordance with the invention can in particular be a modified starch selected from pre-cooked starches; hydrolysed starches; crosslinked starches, for example crosslinked by a methylurea derivative, by octenylsuccinic anhydride or by epichlorohydrin; esterified starches; etherified starches; oxidised starches; refined starches; starches grilled in the presence of acid; grafted starches, for example grafted with sodium polyacrylates; coated starches, for example coated with amino acids; and mixtures thereof.

In particular, a modified starch in accordance with the invention can be a crosslinked starch, and more particularly a starch crosslinked by octenylsuccinic anhydride.

The modified starches are represented, for example, by the products sold under the names C*Tex-Instant (pregelatinized adipate), C*StabiTex-Instant (pregelatinized phosphate), C*PolarTex-Instant (pregelatinized hydroxypropyl), C*Set (acid hydrolysis, oxidation), C*size (oxidation), C*BatterCrisp (oxidation), C*DrySet (dextrinization), C*Tex™ (acetyl distarch adipate), C*PolarTex™ (hydroxypropyl distarch phosphate), C*StabiTex™ (distarch phosphate, acetyl distarch phosphate) by Cargill, by distarch phosphates or compounds rich in distarch phosphate such as the product sold under the references Prejel VA-70-T AGGL (gelatinized hydroxypropyl cassava distarch phosphate) or Prejel TK1 (gelatinized cassava distarch phosphate) or Prejel 200 (gelatinized acetyl cassava distarch phosphate 3025100 14) by Avebe or Structure Zea from National Starch (gelatinized corn distarch phosphate).

A composition according to the invention may comprise a plurality of starches in accordance with the invention, it being possible for these starches to be native and/or modified.

A composition according to the invention can comprise at least 1% by weight, in particular from 1% to 5% by weight, particularly from 1.5% to 4% by weight, and more particularly from 2% to 4% by total weight of starch relative to the total weight of the composition.

“Total weight of starch” means the weight of all the starches in accordance with the invention that are present in a composition of the invention.

In particular, as native or modified starches which may be suitable for the invention, mention may for example be made of those indicated in Table 2 below.

TABLE 2

| Customary chemical | | | | |
|---|---|---|---|---|
| name | Particle size | Chemical family | Trade name | Supplier |
| CORN STARCH | 14.9 μm | Carbohydrates | Dry Flo Plus | NOURYON |
| ESTERIFIED WITH | | | | |
| OCTENYLSUCCINIC | | | | |
| ANHYDRIDE, | | | | |
| AMMONIUM SALT | | | | |
| CORN STARCH: | 14.4 μm | Carbohydrates | Beauté by | ROQUETTE |
| AMYLPECTIN/AMYLOSE | | | Roquette | |
| | | | ST005 | |
| WAXY CORN STARCH | 7-20 μm | Carbohydrates | Star Design | CARGILL |
| | | | Pure | |

As previously indicated, the one or more native or modified starch(es) of a composition according to the invention may in particular be different from the one or more filler(s) which are in particular organic, having an oil absorption capacity, measured at the wet point, of greater than 10 mL/g of fillers, said fillers being separate from the fillers i), also present in a composition of the invention.

A composition according to the invention can in particular comprise a ratio between the content of filler(s) ii) and the content of spherical fillers i) of between 0.1 and 15, preferably between 1 and 3.

Moreover, a composition according to the invention can in particular comprise a ratio between the content of filler(s) ii) and the sum of the contents of spherical fillers i) and of native or modified starch of less than 1.

In particular, a composition according to the invention may comprise:
a ratio between the content of filler(s) ii) and the content of spherical fillers i) of between 0.1 and 15 and preferably of between 1 and 3; and
a ratio between the content of filler(s) ii) and the sum of the contents of spherical fillers i) and of native or modified starch(es) of less than 1.

### Composition According to the Invention

As indicated previously, a composition according to the invention is an inverse emulsion. A composition in accordance with the invention therefore comprises an aqueous phase and an oily phase.

### Aqueous Phase

The aqueous phase of a composition according to the invention comprises water and optionally a water-soluble solvent.

In the present invention, the term “water-soluble solvent” denotes a compound which is liquid at room temperature and miscible with water (miscibility in water of greater than 50% by weight at 25° C. and atmospheric pressure).

The water-soluble solvents that can be used in a composition of the invention may also be volatile.

Among the water-soluble solvents that may be used in the composition in accordance with the invention, mention may particularly be made of lower monoalcohols containing from 1 to 5 carbon atoms, such as ethanol and isopropanol, C₃ and C₄ ketones and C₂-C₄ aldehydes. The aqueous phase may be present in a composition according to the invention in a content ranging from 5% to 60% by weight, better still from 10% to 55% by weight, preferably from 20% to 50% by weight and in particular from 25% to 45% by weight relative to the total weight of said composition.

### Oily Phase

For the purposes of the invention, an oily phase comprises at least one oil.

The term “oil” means any fatty substance that is in liquid form at room temperature and atmospheric pressure.

An oily phase that is suitable for preparing compositions according to the invention may comprise hydrocarbon-based oils, silicone oils, fluoro oils or non-fluoro oils, or mixtures thereof.

The oils may be volatile or non-volatile.

They may be of animal, plant, mineral or synthetic origin.

For the purposes of the present invention, the term “non-volatile oil” means an oil with a vapour pressure of less than 0.13 Pa.

For the purposes of the present invention, the term “silicone oil” means an oil comprising at least one silicon atom, and particularly at least one Si—O group.

The term “fluoro oil” means an oil comprising at least one fluorine atom.

The term “hydrocarbon-based oil” means an oil mainly containing hydrogen and carbon atoms.

The oils may optionally comprise oxygen, nitrogen, sulfur and/or phosphorus atoms, for example in the form of hydroxyl or acid radicals. For the purposes of the invention, the term “volatile oil” means any oil that is capable of evaporating on contact with the skin in less than one hour, at room temperature and atmospheric pressure. The volatile oil is a volatile cosmetic compound that is liquid at room temperature, particularly having a non-zero vapour pressure, at room temperature and atmospheric pressure, particularly having a vapour pressure ranging from 0.13 Pa to 40 000 Pa (10-3 to 300 mmHg), in particular ranging from 1.3 Pa to 13 000 Pa (0.01 to 100 mmHg) and more particularly ranging from 1.3 Pa to 1300 Pa (0.01 to 10 mmHg).

Volatile Oils The volatile oils may be hydrocarbon-based oils or silicone oils.

Among the volatile hydrocarbon-based oils containing from 8 to 16 carbon atoms, mention may particularly be made of branched C8-C16 alkanes, for instance C8-C16 isoalkanes (also known as isoparaffins), isododecane, isodecane, isohexadecane and, for example, the oils sold under the trade names Isopar or Permethyl, branched C8-C16 esters, for instance isohexyl neopentanoate, and mixtures thereof. Preferably, the volatile hydrocarbon-based oil or oils are selected from volatile hydrocarbon-based oils containing from 8 to 16 carbon atoms, and mixtures thereof, in particular from isododecane, isodecane and isohexadecane, and is particularly isohexadecane.

Mention may also be made of volatile linear alkanes comprising from 8 to 16 carbon atoms, in particular from 10 to 15 carbon atoms and more particularly from 11 to 13 carbon atoms, for instance n-dodecane (C12) and n-tetradecane (C14) sold by Sasol under the respective references Parafol 12-97 and Parafol 1497, and also mixtures thereof, the undecane-tridecane mixture, mixtures of n-undecane (C11) and of n-tridecane (C13) obtained in examples 1 and 2 of the application WO 2008/155 059 from Cognis, and mixtures thereof.

Volatile silicone oils that may be mentioned include volatile linear silicone oils such as hexamethyldisiloxane, octamethyltrisiloxane, decamethyltetrasiloxane, tetradecamethylhexasiloxane, hexadecamethylheptasiloxane and dodecamethylpentasiloxane.

Volatile cyclic silicone oils that may be mentioned include hexamethylcyclotrisiloxane, octamethylcyclotetrasiloxane, cyclohexadimethylsiloxane, decamethylcyclopentasiloxane and dodecamethylcyclohexasiloxane.

### Non-Volatile Oils

The composition according to the invention may comprise a non-volatile oil.

It falls within the competence of those skilled in the art to select the nature and amount of non-volatile oil(s) that may be introduced into the composition without impairing the properties thereof.

When they are present, the non-volatile oils may particularly be selected from non-volatile hydrocarbon-based oils, fluoro oils and/or silicone oils. Non-volatile hydrocarbon-based oils that may particularly be mentioned include:
hydrocarbon-based oils of animal origin; hydrocarbon-based oils of plant origin, synthetic ethers containing from 10 to 40 carbon atoms, such as dicaprylyl ether, synthetic esters, such as oils of formula R11COOR12 in which R11 represents a linear or branched fatty acid residue comprising from 1 to 40 carbon atoms and R12 represents a hydrocarbon-based chain, particularly that is branched, containing from 1 to 40 carbon atoms, provided that R11+R12≥10. The esters may for example be selected from esters of alcohol and fatty acid, for example cetostearyl octanoate, isopropyl alcohol esters, such as isopropyl myristate or isopropyl palmitate, ethyl palmitate, 2-ethylhexyl palmitate, isopropyl stearate, octyl stearate, hydroxylated esters, such as isostearyl lactate, octyl hydroxystearate, alcohol or polyalcohol ricinoleates, hexyl laurate, neopentanoic acid esters, such as isodecyl neopentanoate, isotridecyl neopentanoate, isononanoic acid esters, such as isononyl isononanoate, isotridecyl isononanoate, polyol esters and pentaerythritol esters, such as dipentaerythrityl tetrahydroxystearate/tetraisostearate, fatty alcohols that are liquid at room temperature, with a branched and/or unsaturated carbon-based chain containing from 12 to 26 carbon atoms, such as 2-octyldodecanol, isostearyl alcohol, oleyl alcohol, higher C12-C22 fatty acids such as oleic acid, linoleic acid or linolenic acid, and mixtures thereof, non-phenyl silicone oils, for instance caprylyl methicone, and phenyl silicone oils, for instance phenyl trimethicones, phenyl dimethicones, phenyltrimethylsiloxydiphenylsiloxanes, diphenyl dimethicones, diphenylmethyldiphenyltrisiloxanes and 2-phenylethyl trimethylsiloxysilicates, dimethicones or phenyl trimethicone with a viscosity of less than or equal to 100 cSt, trimethylpentaphenyltrisiloxane, and mixtures thereof; and also mixtures of these various oils.

A composition according to the invention may comprise from 1% to 95% by weight, better still 1.5% to 40% by weight, preferably from 2% to 35% by weight of oil(s) relative to the total weight of said composition.

Moreover, since a composition according to the invention is an emulsion, it therefore comprises one or more surfactant(s).

Examples of surfactants that may be mentioned include dimethicone copolyols, such as the mixture of cyclomethicone and dimethicone copolyol sold under the name DC 5225° C. by Dow Corning, and alkyl dimethicone copolyols such as the lauryl methicone copolyol sold under the name Dow Corning 5200 Formulation Aid by Dow Corning, and the cetyl dimethicone copolyol sold under the name Abil EM 90® by Goldschmidt.

A composition according to the invention may particularly comprise, as surfactant, a mixture of crosslinked polyalkylene polydimethylsiloxane and polydimethylsiloxane. A composition according to the invention may comprise, as co-surfactant, lauryl PEG-9 polydimethylsiloxyethyl dimethicone. A composition according to the invention may in particular comprise, as surfactants, a mixture of crosslinked polyalkylene polydimethylsiloxane and polydimethylsiloxane, and lauryl PEG-9 polydimethylsiloxyethyl dimethicone.

A composition according to the invention may comprise an amount of the surfactant(s) at a content of between 0.1% and 50% by weight, in particular between 0.5% and 30% by weight, relative to the total weight of the emulsion.

Moreover, a composition according to the invention advantageously comprises less than 0.1% by weight, in particular less than 0.05% by weight, relative to the total weight of the composition, particularly less than 0.01% by weight, and more particularly is free (comprises 0% by weight), of “microplastic particles”, also referred to in the present text as “microplastics”.

There is no internationally recognized definition of the terms “microplastic particles” or “microplastics”. Generally speaking, they are solid plastic particles that are not water-soluble and are of a size smaller than or equal to five millimetres, and are not biodegradable (according to the definition given by the UNEP).

A composition according to the invention may also comprise at least one preservative. The term “preservative” or “preserving agent” means any cosmetically or pharmaceutically acceptable compound which can prevent microbial growth (or the growth of microorganisms) which may take place in cosmetic or pharmaceutical compositions, from the moment of their preparation, while they are in storage and up to the time of their conventional use by consumers. Preserving agents that may particularly be mentioned include the preserving agents described in Cosmetics, Kirk-Othmer Encyclopedia of Chemical Technology. John Wiley & Sons, Inc., Martin M. Rieger; 5.2 preservatives & table 3, Apr. 12, 2000, https://doi.org/10.1002/0471238961.0315191318090507.a01.

As examples of preserving agents that are suitable for use in the invention, mention may be made of esters of para-hydroxybenzoic acid, also known as parabens (in particular methyl paraben, ethyl paraben, propyl paraben), phenoxyethanol, formaldehyde releasers, for instance imidazolidinylurea or diazolidinylurea, chlorhexidine digluconate, alkali metal or alkaline-earth metal benzoates, such as sodium benzoate, caprylyl glycol, iodopropynyl butyl carbamate, pentylene glycol, alkyltrimethylammonium bromide such as myristyltrimethylammonium bromide (CTFA name: Myrtrimonium bromide), dodecyltrimethylammonium bromide, hexadecyltrimethylammonium bromide, and mixtures thereof, more preferentially alkali metal or alkaline-earth metal benzoates, in particular alkali metal benzoates, such as sodium benzoate.

The composition according to the invention may also comprise at least one wax at room temperature. For the purposes of the present invention, the term “wax” means a lipophilic compound, which is solid at room temperature (25° C.), with a reversible solid/liquid change of state, with a melting point of greater than or equal to 30° C., which may be up to 120° C. By bringing the wax to the liquid state (melting), it is possible to make it miscible with the oils that are optionally present and to form a microscopically homogeneous mixture, but on returning the temperature of the mixture to room temperature, recrystallization of the wax in the oils of the mixture is obtained. The melting point of the wax may be measured using a differential scanning calorimeter (D.S.C.), for example the calorimeter sold under the name DSC 30 by Mettler. The waxes may be hydrocarbon-based waxes, fluoro waxes and/or silicone waxes, and may be of plant, mineral, animal and/or synthetic origin. In particular, the waxes have a melting point of greater than 25° C. and better still greater than 45° C. As waxes that may be used in the composition of the invention, mention may be made of beeswax, carnauba wax or candelilla wax, paraffin, microcrystalline waxes, ceresin or ozokerite; synthetic waxes such as polymethylene waxes and polyethylene waxes or Fischer-Tropsch waxes, and silicone waxes such as alkyl or alkoxy dimethicones containing from 16 to 45 carbon atoms.

A composition according to the invention may also contain one or more adjuvants that are customary in cosmetics, such as agents for preventing hair loss; oxidizing agents; vitamins and provitamins; sunscreens; colorants; nacres and/or opacifiers; sequestrants; plasticizers; antioxidants; hydroxy acids; such as a-hydroxy acids; fixing polymers; fillers; propellants; antiwrinkle agents; soothing agents; plant extracts; vegetable proteins and hydrolyzates thereof such as rice or soybean protein hydrolyzates; algal extracts, in particular Laminaria extracts; oligopeptides and pseudodipeptides and the acylated derivatives thereof; hyaluronic acid fragments; sphingosines, such as saliciloyl phytosphingosine; urea and derivatives thereof, such as 2-hydroxyethylurea; monosaccharides, such as mannose; hyaluronic acid; AHAs; BHAs; acrylic acid homopolymers, ceramides; ceramide derivatives; cholesterol sulfates; fatty acids; and mixtures thereof, these adjuvants being different from the fillers i) and ii) of the invention.

The amounts of these various adjuvants are those conventionally used in the fields under consideration.

Needless to say, those skilled in the art will take care to select this or these optional compounds to be added to a composition according to the inventions such that the advantageous properties intrinsically associated with the composition in accordance with the invention are not, or are not substantially, adversely affected by the envisioned addition(s).

In particular, a composition according to the invention, in the form of an inverse emulsion, comprises:
i) at least 0.05% by weight, relative to the total weight of the composition, in particular from 0.05% to 2% by weight, more particularly from 0.05% to 1% by weight, particularly from 0.05% to 0.5% by weight, in particular from 0.05% to 0.2% by weight, and more particularly from 0.1% to 0.2% by weight of spherical filler(s) having a mean diameter ranging from 1 to 100 μm, in particular ranging from 1 to 50 μm, more particularly less than 30 μm, and particularly of between 1 and 25 μm;
ii) at least 0.1% by weight, relative to the total weight of the composition, in particular from 0.1% to 2% by weight, particularly from 0.1% to 1% by weight, and more particularly from 0.2% to 0.8% by weight of filler(s) having an oil absorption capacity, measured at the wet point, of greater than 10 ml/g of fillers, said fillers being separate from the fillers i);
iii) at least 1% by weight, in particular from 1% to 5% by weight, particularly from 1.5% to 4% by weight, and more particularly from 2% to 4% by total weight of native or modified starch(es); and
iv) less than 0.1% by weight, in particular less than 0.05% by weight, particularly less than 0.01% by weight, and more particularly 0% by weight of microplastic particles relative to the total weight of the composition.

In particular, a composition according to the invention, in the form of an inverse emulsion, comprises:
i) at least 0.05% by weight, relative to the total weight of the composition, in particular from 0.05% to 2% by weight, more particularly from 0.05% to 1% by weight, particularly from 0.05% to 0.5% by weight, in particular from 0.05% to 0.2% by weight, and more particularly from 0.1% to 0.2% by weight of spherical filler(s) having a mean diameter ranging from 1 to 100 μm, in particular ranging from 1 to 50 μm, more particularly less than 30 μm, and particularly of between 1 and 25 μm;
ii) at least 0.1% by weight, relative to the total weight of the composition, in particular from 0.1% to 2% by weight, particularly from 0.1% to 1% by weight, and more particularly from 0.2% to 0.8% by weight of filler(s) having an oil absorption capacity, measured at the wet point, of greater than 10 ml/g of fillers, said fillers being separate from the fillers i); and
iii) at least 1% by weight, in particular from 1% to 5% by weight, particularly from 1.5% to 4% by weight, and more particularly from 2% to 4% by total weight of native or modified starch(es);
the ratio between the content of filler(s) ii) and the content of spherical fillers i) is between 0.1 and 15 and preferably between 1 and 3;
iv) less than 0.1% by weight, in particular less than 0.05% by weight, particularly less than 0.01% by weight, and more particularly 0% by weight of microplastic particles relative to the total weight of the composition; and
the ratio between the content of filler(s) ii) and the sum of the contents of spherical fillers i) and of native or modified starch(es) is less than 1.

In particular, a composition according to the invention, in the form of an inverse emulsion, comprises:
i) at least 0.05% by weight, relative to the total weight of the composition, in particular from 0.05% to 2% by weight, more particularly from 0.05% to 1% by weight, particularly from 0.05% to 0.5% by weight, in particular from 0.05% to 0.2% by weight, and more particularly from 0.1% to 0.2% by weight of spherical filler(s) having a mean diameter ranging from 1 to 100 μm, in particular ranging from 1 to 50 μm, more particularly less than 30 μm, and particularly of between 1 and 25 μm, selected from the group consisting of cellulose beads and perlite;
ii) at least 0.1% by weight, relative to the total weight of the composition, in particular from 0.1% to 2% by weight, particularly from 0.1% to 1% by weight, and more particularly from 0.2% to 0.8% by weight of filler(s) having an oil absorption capacity, measured at the wet point, of greater than 10 ml/g of fillers, said fillers being particles of silica aerogel treated with hexamethyldisiloxane;
iii) at least 1% by weight, in particular from 1% to 5% by weight, particularly from 1.5% to 4% by weight, and more particularly from 2% to 4% by total weight of native or modified starch(es), the starch being native or modified corn starch, in particular modified corn starch, more particularly corn starch crosslinked by octenylsuccinic anhydride; and
iv) less than 0.1% by weight, in particular less than 0.05% by weight, particularly less than 0.01% by weight, and more particularly 0% by weight of microplastic particles relative to the total weight of the composition.

### Intended Use of a Composition According to the Invention

As indicated previously, a composition according to the invention is preferably a cosmetic composition, generally suitable for topical application to keratin materials, in particular the skin, and particularly the skin of the face and/or body, more particularly of the face, and therefore generally comprising a physiologically acceptable medium.

A composition according to the invention may be in any presentation form conventionally used in the field of cosmetics according to the intended applications, in particular for topical application.

In particular, a composition according to the invention may be in the form of a fluid emulsion, a cream, a lotion, a milk or a serum.

A composition according to the invention may be obtained by any method known to those skilled in the art for the formulation of cosmetic compositions for the care and/or hygiene of keratin materials, in particular of care cream or cleansing cream type. It is within the competence of a person skilled in the art to adapt the conditions for preparing a composition according to the invention.

A composition according to the invention may be used on all keratin materials, particularly as a hygiene product, for example as a product for cleansing and/or removing makeup from the skin, mucous membranes and/or hair, in particular as a product for caring for and/or cleansing and/or removing makeup from the skin.

Another subject of the invention is therefore a cosmetic process for caring for and/or cleansing keratin materials, in particular the skin, and more particularly facial skin, comprising the application, to said keratin materials, of a composition according to the invention.

The invention is illustrated in greater detail by the examples presented below. Unless otherwise indicated, the amounts indicated are expressed as mass percentages.

### EXAMPLES

### Example 1

A reference composition of water-in-silicone inverse emulsion cream comprising 0.2% microspheres (vinylidene chloride/acrylonitrile/methyl methacrylate copolymer) expanded by isobutane is produced and is illustrated in Table 1 below.

A composition in accordance with the invention is also formulated (formula 1 in Table 3 below), which is identical to the reference composition (not in accordance with the invention) but in which the microspheres have been replaced by cellulose beads and silica aerogel.

Three comparative compositions (counter-examples) 1, la and 1b are also formulated, and differ from composition 1 according to the invention by the absence of cellulose beads 5 (counter-example 1), the absence of cellulose beads and silica aerogel (counter-example 2) or the absence of modified starch (counter-example 3), respectively.

TABLE 3

| | Reference | | Counter- | Counter- | Counter- |
|---|---|---|---|---|---|
| | composition | | example 1 | example 1a | example 1b |
| | (not in | Composition | (not in | (not in | (not in |
| | accordance | 1 (according | accordance | accordance | accordance |
| | with the | to the | with the | with the | with the |
| Chemical name | invention) | invention) | invention) | invention) | invention) |
| Mixture of crosslinked | 15% | 15% | 15% | 15% | 15% |
| polyalkylene | | | | | |
| polydimethylsiloxane and | | | | | |
| polydimethylsiloxane | | | | | |
| Cyclohexamethylsiloxane | 10% | 10% | 10% | 10% | 10% |
| Polymethylene wax | 4% | 4% | 4% | 4% | 4% |
| Lauryl PEG-9 | 1% | 1% | 1% | 1% | 1% |
| polydimethylsiloxyethyl | | | | | |
| dimethicone | | | | | |
| Microbiological-grade | 30.8% | 30.7% | 30.8% | 31% | 33.2% |
| deionized water | | | | | |
| Ethylenediaminetetraacetic | 0.1% | 0.1% | 0.1% | 0.1% | 0.1% |
| acid, disodium salt | | | | | |
| Glycerol | 4% | 4% | 4% | 4% | 4% |
| 2-phenoxyethanol | 0.7% | 0.7% | 0.7% | 0.7% | 0.7% |
| Magnesium sulfate | 0.7% | 0.7% | 0.7% | 0.7% | 0.7% |
| C-beta-D-xylopyranoside-2- | 30% | 30% | 30% | 30% | 30% |
| hydroxypropane dissolved at | | | | | |
| approximately 35% in a | | | | | |
| 60/40 water/1,2- | | | | | |
| propanediol mixture | | | | | |
| Microspheres (vinylidene | 0.2% | 0% | 0% | 0% | 0% |
| chloride/acrylonitrile/methyl | | | | | |
| methacrylate copolymer) | | | | | |
| expanded by isobutane | | | | | |
| (EXPANCEL ® 551 DE 40 D42) | | | | | |
| Corn starch esterified by | 2.5% | 2.5% | 2.5% | 2.5% | 0% |
| octenylsuccinic anhydride, | | | | | |
| aluminium salt (Dry Flo Plus, | | | | | |
| Nouryon) | | | | | |
| Nacre | 1% | 1% | 1% | 1% | 1% |
| Silica aerogel treated with | 0% | 0.2% | 0.2% | 0% | 0.2% |
| hexamethyldisiloxane | | | | | |
| (without loose powder) | | | | | |
| (DOWSIL VM-2270, Dow | | | | | |
| Corning) | | | | | |
| Cellulose beads | 0% | 0.1% | 0% | 0% | 0.1% |
| (CELLULOBEADS USF, Kobo) | | | | | |

The percentages are indicated as percentage by weight relative to the total weight of the composition.

All these compositions are evaluated by a panel of 20 experts.

### Evaluation of Composition 1 According to the Invention

First of all, composition 1 according to the invention is evaluated in comparison with the reference composition.

The comparison of these evaluations shows that the sensoriality is identical in terms of appearance and handling of the cream (shine, smoothness, transparency, fluidity, wobble, tack on the fingers, handling, consistency, shape holding when handled, and thickness) and also in terms of application qualities (whiteness of the film, greasy feel) and in terms of skin finish.

Replacing the microplastic particles with the combination of ingredients according to the invention does indeed make it possible to retain the sensorial and application properties of a cosmetic composition.

### Evaluation of the Composition According to Counter—Example 1

As indicated above, the composition of counter-example 1 corresponds to the reference composition in which, aside from removing the microspheres and in addition to the starch, only 0.2% by weight of silica aerogel is introduced, with no spherical fillers in accordance with the invention.

The panel evaluates the formula according to the Reference composition and the formula prepared according to counter-example 1. Compared to the formula according to the Reference composition, counter-example 1 is considered to drag more when spread over the skin and to give a rougher feel in terms of skin finish.

This shows the need to have, in addition to the starch, the combination of two types of fillers according to the invention, with one alone being insufficient to obtain a composition that retains the sensorial and application properties of a cosmetic composition comprising microplastic particles.

### Evaluation of the Composition According to Counter—Example 1a

As indicated above, the composition of counter-example 1a corresponds to the reference composition in which, aside from removing the microspheres, only starch is present. Neither of the two fillers in accordance with the invention is introduced.

The panel evaluates the formula according to the Reference composition and the formula prepared according to counter-example 1a. Compared to the formula according to the Reference composition, counter-example 1a is considered to be too slippery when spread over the skin and to give a skin finish which is too greasy and too shiny.

This shows the need to have a combination of two types of fillers in accordance with the invention.

### Evaluation of the Composition According to Counter—Example 1b

As indicated above, the composition of counter-example 1b corresponds to the reference composition in which, aside from removing the microspheres, the starch is not introduced, while silica aerogel and spherical fillers in accordance with the invention are present. The panel evaluates the formula according to the Reference composition and the formula prepared according to counter-example 1b. Compared to the formula according to the Reference composition, counter-example 1b is too slippery under the finger when holding and applying to the skin, which gives a feeling of a greasier cream.

This shows the need to introduce starch in addition to the combination of spherical fillers+fillers having a high oil absorption capacity in accordance with the invention.

### Example 2

The reference composition of water-in-silicone inverse emulsion cream of example 1 is compared to a composition in accordance with the invention (example 2 Composition) in which the microplastic fillers are replaced with a combination of silica aerogel and spherical expanded perlite 25 μm in diameter, in addition to the presence of modified starch (see Table 4 below).

TABLE 4

| | Reference | |
|---|---|---|
| | composition | Composition |
| | (not in | Example 2 (in |
| | accordance | accordance |
| | with the | with the |
| Chemical name | invention) | invention) |
| Mixture of crosslinked | 15% | 15% |
| polyalkylene | | |
| polydimethylsiloxane and | | |
| polydimethylsiloxane | | |
| Cyclohexamethylsiloxane | 10% | 10% |
| Polymethylene wax | 4% | 4% |
| Lauryl PEG-9 | 1% | 1% |
| polydimethylsiloxyethyl | | |
| dimethicone | | |
| Microbiological-grade | 30.8% | 30.5% |
| deionized water | | |
| Ethylenediaminetetraacetic | 0.1% | 0.1% |
| acid, disodium salt | | |
| Glycerol | 4% | 4% |
| 2-phenoxyethanol | 0.7% | 0.7% |
| Magnesium sulfate | 0.7% | 0.7% |
| C-beta-D-xylopyranoside-2- | 30% | 30% |
| hydroxypropane dissolved at | | |
| approximately 35% in a 60/40 | | |
| water/1,2-propanediol mixture | | |
| Microspheres (vinylidene | 0.2% | 0% |
| chloride/acrylonitrile/methyl | | |
| methacrylate copolymer) | | |
| expanded by isobutane | | |
| (EXPANCEL ® 551 DE 40 D42) | | |
| Corn starch esterified by | 2.5% | 2.5% |
| octenylsuccinic anhydride, | | |
| aluminium salt (Dry Flo Plus, | | |
| NOURYON) | | |
| Nacre | 1% | 1% |
| Silica aerogel treated with | 0% | 0.2% |
| hexamethyldisiloxane (without | | |
| loose powder) (DOWSIL VM- | | |
| 2270, AEROGEL FINE PARTICLES) | | |
| Perlite (Optimat 2550 OR, | 0% | 0.2% |
| IMERYS) | | |

The percentages are indicated as percentage by weight relative to the total weight of the composition.

### Evaluation of the Composition According to Example 2

Sensory analysis is conducted by a panel constituted of 20 experts.

The panel evaluates the formula according to the Reference composition and the composition formulated according to Example 2. The comparison of these evaluations shows that the sensoriality is identical in terms of appearance and handling of the cream (shine, smoothness, transparency, fluidity, wobble, tack on the fingers, handling, consistency, shape holding when handled, and thickness) and also in terms of application qualities (whiteness of the film, greasy feel) and in terms of skin finish.

1. Composition, preferably cosmetic composition, particularly for caring for or cleansing keratin materials, in particular the skin, more particularly facial skin, comprising:
i) one or more spherical filler(s) which are organic or inorganic, in particular organic, having a mean diameter ranging from 1 to 100 μm;
ii) one or more filler(s) which are in particular organic, having an oil absorption capacity, measured at the wet point, of greater than 10 ml/g of fillers, said fillers being separate from the fillers i); and
iii) one or more native or modified starch(es);
said composition being an inverse emulsion,
wherein the spherical fillers i) are selected from the group consisting of cellulose beads;
glass microbeads; N-lauroyl L-lysine, and perlite, in particular expanded perlite, the mean diameter being the mean diameter on 50% by volume of the particles (D [0,5]).

2. Composition according to claim 1, characterized in that it comprises less than 0.1% by weight, in particular less than 0.05% by weight, particularly less than 0.01% by weight, and more particularly 0% by weight of microplastic particles relative to the total weight of the composition.

3. Composition according to claim 1 or 2, comprising at least 0.05% by weight, in particular from 0.05% to 2% by weight, more particularly from 0.05% to 1% by weight, particularly from 0.05% to 0.5% by weight, in particular from 0.05% to 0.2% by weight, and more particularly from 0.1% to 0.2% by weight of spherical fillers i) relative to the total weight of the composition.

4. Composition according to any one of claims 1 to 3, comprising at least 0.1% by weight, in particular from 0.1% to 2% by weight, particularly from 0.1 to 1% by weight, and more particularly from 0.2% to 0.8% by weight of fillers ii) relative to the total weight of the composition.

5. Composition according to any one of claims 1 to 4, comprising at least 1% by weight, in particular from 1% to 5% by weight, particularly from 1.5% to 4% by weight, and more particularly from 2% to 4% by total weight of starch relative to the total weight of the composition.

6. Composition according to any one of claims 1 to 5, wherein the spherical fillers i) are selected from the group consisting of cellulose beads and perlite.

7. Composition according to any one of claims 1 to 6, wherein the fillers ii) are silica aerogel particles and in particular hydrophobic silica aerogel particles, particularly are particles of silica aerogel treated with hexamethyldisiloxane.

8. Composition according to any one of claims 1 to 7, wherein the native or modified starch is different from the filler(s) (ii), in particular is different from the filler(s) (i) and the fillers (ii).

9. Composition according to any one of claims 1 to 8, wherein the native or modified starch is selected from starches of corn, rice, cassava, potato, wheat, sorghum, pea, and mixtures thereof, and is in particular corn starch.

10. Composition according to any one of claims 1 to 9, wherein the modified starch is selected from pre-cooked starches; hydrolysed starches; crosslinked starches, for example crosslinked by a methylurea derivative, by octenylsuccinic anhydride or by epichlorohydrin; esterified starches; etherified starches; oxidised starches; refined starches; starches grilled in the presence of acid; grafted starches, for example grafted with sodium polyacrylates; coated starches, for example coated with amino acids; and mixtures thereof, preferably selected from crosslinked starches, and particularly starches crosslinked by octenylsuccinic anhydride.

11. Composition according to any one of claims 1 to 10, wherein the ratio between the content of filler(s) ii) and the content of spherical fillers i) is between 0.1 and 15, in particular between 0.5 and 8, and preferably between 1 and 3.

12. Composition according to any one of claims 1 to 11, wherein the ratio between the content of filler(s) ii) and the sum of the contents of spherical fillers i) and of native or modified starch(es) is less than 1.

13. Composition according to any one of claims 1 to 12, comprising:
i) at least 0.05% by weight, relative to the total weight of the composition, in particular from 0.05% to 2% by weight, more particularly from 0.05% to 1% by weight, particularly from 0.05% to 0.5% by weight, in particular from 0.05% to 0.2% by weight, and more particularly from 0.1% to 0.2% by weight of spherical filler(s) having a mean diameter ranging from 1 to 100 μm, in particular ranging from 1 to 50 μm, more particularly less than 30 μm, and particularly of between 1 and 25 μm, selected from the group consisting of cellulose beads and perlite;
ii) at least 0.1% by weight, relative to the total weight of the composition, in particular from 0.1% to 2% by weight, particularly from 0.1% to 1% by weight, and more particularly from 0.2% to 0.8% by weight of filler(s) having an oil absorption capacity, measured at the wet point, of greater than 10 ml/g of fillers, said fillers being particles of silica aerogel treated with hexamethyldisiloxane;
iii) at least 1% by weight, in particular from 1% to 5% by weight, particularly from 1.5% to 4% by weight, and more particularly from 2% to 4% by total weight of native or modified starch(es), the starch being native or modified corn starch, in particular modified corn starch, more particularly corn starch crosslinked by octenylsuccinic anhydride; and
iv) less than 0.1% by weight, in particular less than 0.05% by weight, particularly less than 0.01% by weight, and more particularly 0% by weight of microplastic particles relative to the total weight of the composition.

14. Cosmetic process for caring for and/or cleansing keratin materials, in particular the skin, and more particularly facial skin, comprising the application, to said keratin materials, of a cosmetic composition according to any one of claims 1 to 13.
